(19) United States
(12) Patent Application Publication
(10) Pub. No.: US 2021/0145363 A1
(43) Pub. Date: May 20, 2021

(54) MEASURING DEVICE

(71) Applicant: Ingo FLORE, Dortmund (DE)

(72) Inventors: Ok-Kyung CHO, Schwerte (DE); Yoon Ok KIM, Schwerte (DE)

(21) Appl. No.: 17/254,658

(22) PCT Filed: Jun. 24, 2019
(86) PCT No.: PCT/EP2019/066615

§ 371 Date: Dec. 21, 2020

(30) Foreign Application Priority Data
Jun. 22, 2018 (DE) 10 2018 004 955.9

(51) Int. Cl. A61B 5/00; A61B 5/0205

(52) U.S. Cl. CPC A61B 5/6826; A61B 5/02055; A61B 5/14552; A61B 5/6838; A61B 5/6843

(57) ABSTRACT

The invention relates to a multifunctional measuring device comprising a housing (1) having an upper shell (2) and a lower shell (3), which are movable relative to one another by means of a hinge mechanism (4) and comprise cavities which correspond to one another, wherein the cavities form a chamber (9) accessible from the outside for receiving a human finger, wherein an optical measuring unit having an optical module (11), which comprises at least one light source (12) and at least one sensor, is arranged in the chamber (9), and means for data evaluation and/or data transfer are integrated in or on the housing. The aim of the invention is to develop a compact, easy-to-handle measuring device of this kind such that it is possible to determine a variety of parameters that can be determined non-invasively by means of the measuring device. Furthermore, statistical methods are intended to be used to make it possible to determine additional parameters that are normally not directly accessible to the non-invasive measurement. To do this, the invention proposes that different sensor systems are integrated in the compact measuring device, in the chamber (9) and/or on the outside of the housing (1).

The invention relates to a multifunctional measuring device comprising a housing having an upper shell and a lower shell, which are movable relative to one another by means of a hinge mechanism and comprise cavities which correspond to one another, wherein the cavities form a chamber accessible from the outside for receiving a human finger, wherein an optical measuring unit having an optical module, which comprises at least one light source and at least one sensor, is arranged in the chamber, and wherein means for data evaluation and/or data transfer are integrated in or on the housing. Furthermore, the invention relates to a method for carrying out a measurement using a multifunctional measuring apparatus of this kind.

Portable, easy-to-use multifunctional measuring devices for healthcare and medical applications allow users to monitor their state of health both at home and out and about. Depending on the scope of application and the purpose, different parameters may be relevant for monitoring the user's state of health, for example heart rate, arterial oxygen saturation, or other parameters derived from an ECG (electrocardiogram) or photoplethysmogram.

Measuring devices, known as “finger pulse oximeters”, are often used to measure pulse and oxygen saturation.

If, however, a plurality of additional, different physiological parameters are intended to be determined, different individual devices often have to be used. This is impractical for the user, both in terms of purchasing and usage. In addition, when using different devices, it is complicated to integrate and combine the measured data.

The object of the invention is therefore to develop a compact, easy-to-handle measuring device such that it is possible to determine a variety of parameters that can be determined non-invasively by means of the measuring device. Furthermore, statistical methods (e.g. multivariate methods) and/or machine-learning methods (e.g. neural networks, also in connection with deep learning) are intended to be used to make it possible to determine additional parameters that are normally not directly accessible to the non-invasive measurement.

To achieve the object, proceeding from a measuring device of the type mentioned at the outset, the invention proposes that at least one electrical measuring unit is provided, comprising at least two measuring electrodes in the chamber and/or on the outside of the housing. In addition to the optical measurements, the electrical measuring unit can also be used to carry out electrical measurements, such as a bioimpedance measurement or an electrocardiogram measurement (ECG). In addition, the additional electrical measured results can be combined with the optical measured results. This is discussed in greater detail below.

A development of the invention provides that at least one temperature-measuring device is arranged in and/or on the housing. By means of the temperature-measuring unit, the user's finger temperature can be ascertained, and the corresponding measured data can be included in the evaluation.

A preferred embodiment of the invention provides that at least one additional optical sensor and/or one additional light source is arranged opposite the optical module. By arranging an additional sensor or light source, transmission measurements can also be carried out in addition to the reflection measurement by means of the optical sensor and the light source in the optical module, and the thus obtained measured data can be consulted for the analysis. By measuring the reflection and transmission, it is possible to determine physiological parameters for different tissue regions (tissue layers that are closer to the surface or are deeper). Different tissue regions have different venous and/or arterial blood supplies. The combination of measured values from tissue having a venous and/or arterial blood supply makes it possible to draw conclusions on important metabolic parameters.

It is expedient for the hinge mechanism to be provided with a return mechanism. A spring mechanism may be used for this purpose, for example. Once the two shells have been pushed apart and the finger has been inserted, the two shells close again automatically and clamp the finger there-between. By means of the return mechanism, the pressure of the clamping can be preset to the desired value in a reproducible manner. The contact pressure of the finger tissue on the corresponding sensors influences the measurement. This parameter should therefore be defined (at least approximately).

A preferred embodiment provides that a microcontroller is arranged in the housing for data evaluation. By means of the microcontroller, the data evaluation can be carried out directly in the measuring device.

A development of the invention provides that the means for data transfer have a wireless interface. Said interface can transfer the data and the user can view, save and process the data on an external device, such as a smartphone or a smartwatch. It is also possible to control the measuring device by means of an external device of this kind.

It is particularly expedient for devices for positioning the individual fingers to be provided such that the fingers are always in the same position during the measuring process. This can ensure that the fingers are correctly positioned for carrying out the measurement. The specific designs of the respective devices are described in greater detail below.

In an embodiment of the measuring device, it is provided that an accelerometer and/or gyroscope is integrated. As a result, movements of the measuring device can be taken into account in the data evaluation, or the user can be notified that the measured values are potentially incorrect due to movement of the measuring device being too pronounced.

It may also be expedient to integrate additional sensors for measuring the air pressure, humidity and/or the ambient temperature. As a result, the influence of the environmental parameters can be included in the measured-data analysis.

It is also advantageous for pressure sensors to be integrated for measuring the contact pressure of the finger. As a result, a malfunction of the return mechanism can be detected, for example. The measurements by the pressure sensors may, however, also be used for correcting pressure-dependent measured values. In addition, depending on the intended application, it may be useful to evaluate the pressure change overlaid on the contact pressure and caused by blood pulsating in the finger as a separate measured signal and to derive physiological parameters therefrom.

Furthermore, it may be expedient for external connections for additional external sensor systems to be arranged on the housing. External sensors may also be connected to the connections, such that they can be attached to body parts other than the hand, for example.

Embodiments of the invention are explained in greater detail in the following with reference to drawings, in which:

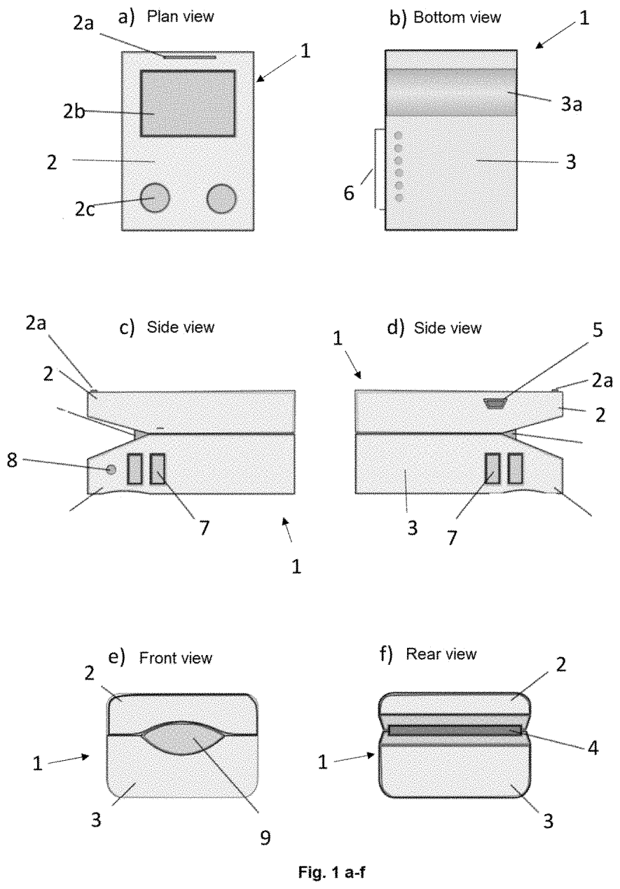

FIG. 1 a-f are various views of a measuring device according to the invention when closed;

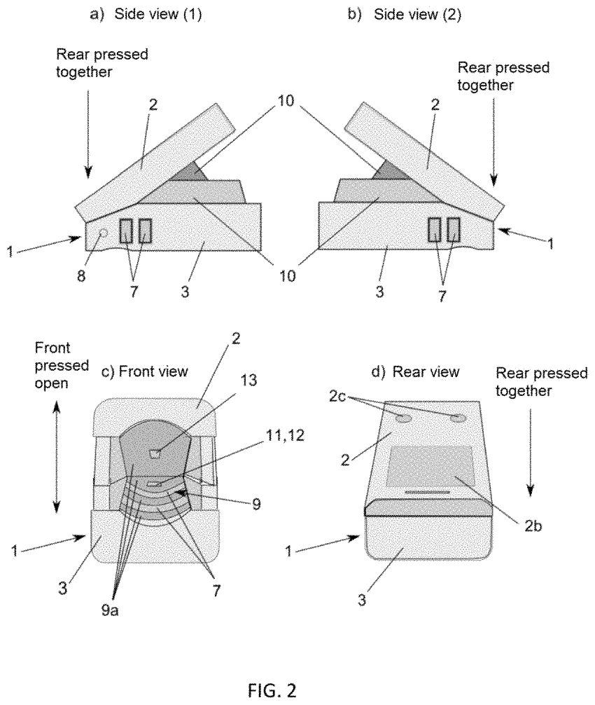

FIG. 2 a-d are various views of a measuring device according to the invention from FIG. 1 a-f when open;

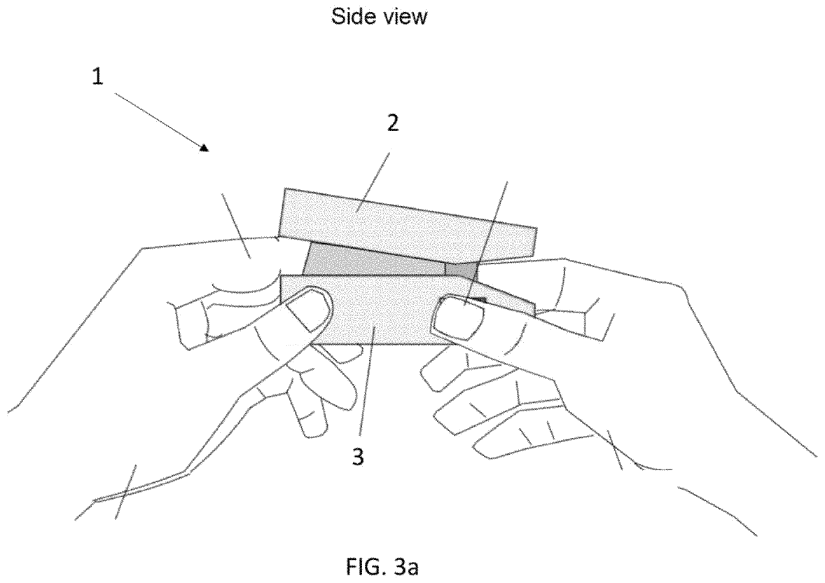

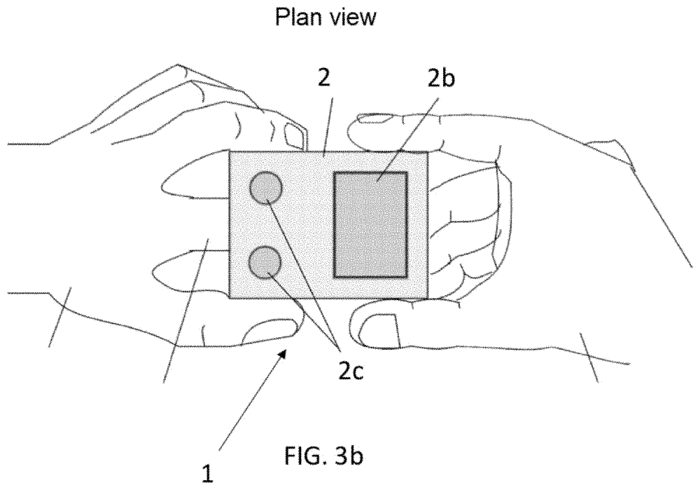

FIG. 3 a-b is a schematic view of a measuring device according to the invention when being used by a user;

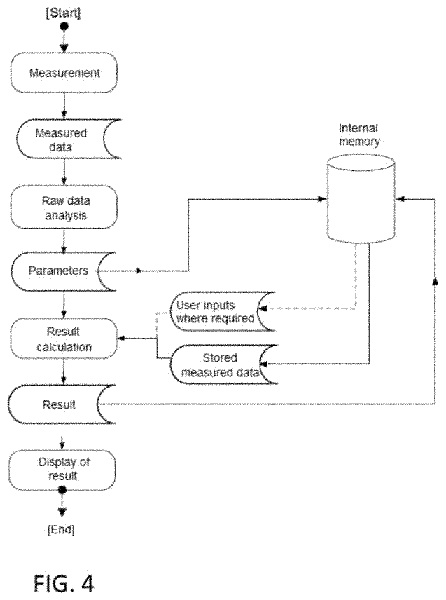

FIG. 4 shows a schematic method sequence during a measurement using a measuring device according to the invention;

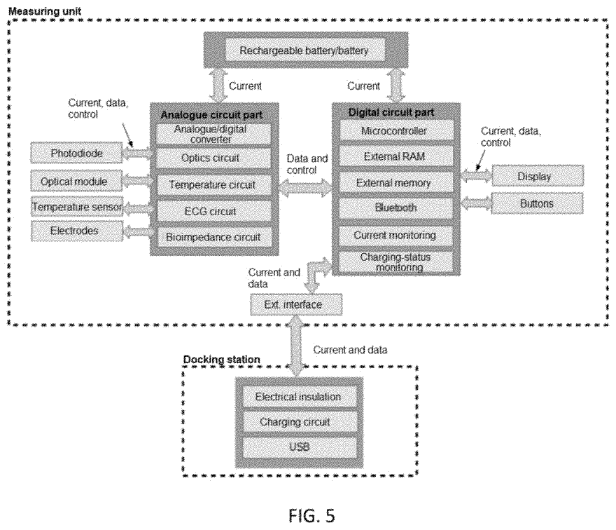

FIG. 5 schematically shows the detection and processing of the measured data.

A measuring device according to the invention is shown in FIG. 1 a-f on the basis of a specific configuration. This view is limited to the external features of the measuring device, with further mechanical aspects and the features of the inner part of the measuring device being described below.

The housing as a whole is denoted by reference sign 1. The essential features of the housing 1 of the measuring device are as follows:
The housing 1 consists of an upper shell 2 and a lower shell 3, which can be moved away from one another at the front by pressing together a hinge mechanism 4 at the rear of the housing 1.
In order to make it easier for the user to grip the measuring device when pressing it together at the rear of the housing 1, there is a small ridge 2a on the upper shell 2 and a depression 3a on the lower shell.
The upper shell 2 contains a display 2b and the control elements 2c. On the side of the upper shell 2, there is optionally a connector 5 for an external interface (e.g. USB) and metal contacts 6, for example for charging the measuring device in a docking station.
There are two electrodes 7 on each side of the lower shell 3 for bioimpedance and ECG measurements. There is additionally a temperature sensor 8 on one of the sides of the housing.
The front view (FIG. 1e) shows a chamber 9, into which a finger can be inserted when said device is open. There is at least one optical measuring unit, and optionally yet more measuring units, in the chamber 9.

FIG. 2 a-d show the upper shell 2 and the lower shell 3 being pressed together at the rear of the measuring device from FIG. 1 a-f. When the upper shell 2 and the lower shell 3 have been opened at the front of the housing 1, a finger can be inserted into the measuring device. The upper shell 2 and lower shell 3 are interconnected by a spring mechanism which acts as a hinge mechanism 4.

When the finger is inserted and the upper shell 2 and lower shell 3 pressed together at the rear part of the measuring device are released, the upper shell 2 and lower shell 3 come together and a defined pressure is exerted on the finger by the spring mechanism. However, other mechanisms that make it possible to open the measuring device in order to insert the finger and exert a defined pressure on the finger are likewise possible and do not affect the core concept of the invention.

FIG. 2 a-d show the measuring device from FIG. 1 a-f when open. Since the upper shell 2 and lower shell 3 cannot move back completely into their starting position when a finger is inserted, laterally attached walls 10, which reduce the incidence of ambient light, are provided both on the upper shell 2 and the lower shell 3. The cavities in the upper shell 2 and lower shell 3, which are shown in FIG. 2c, are located between these walls 10. The cavities form the chamber 9 for receiving a finger. The essential properties of the inner chamber 9 are as follows:
The chamber 9 is curved both at the top and at the bottom. The curvature of the chamber both reflects the curvature of a human finger, and also ensures that the inserted finger is in a stable measuring position.
At the rear end of the chamber 9, there is a rear wall, up to which the finger has to be slid in. This means that the finger has a fixed end position.
Multiple types of measuring unit, which can be used for optical, electrical and temperature measurements, can be integrated in the chamber.
The parts of the contact surface that do not contain measuring units are lined with a soft material 9a, so that sharp edges are prevented and user comfort is increased.

The sensors used here and the position thereof will be discussed in greater detail in the following section.
The lower part of the chamber 9 contains an optical measuring unit in the form of an optical module 11 comprising light sources 12 as well as optical sensors. The optical module 11 is positioned below the distal phalanx. The diffuse reflection of the finger tissue can be measured by means of the optical sensors and the light sources 12 in the optical module 11.
The light sources 12 of the optical module 11 may for example be LEDs having different wavelengths. For example, one or more photodiodes may be used in the optical module 11 for measuring the diffuse reflection of the finger. The module 11 may have an additional temperature sensor, which can provide information relating to the temperature of the light sources 12 within the module 11.
Another optical sensor 13 is positioned in the upper part of the finger support as part of the optical measuring unit. By means of this additional sensor 13, transmission through the finger can be measured, with the irradiated tissue being different compared with the lower sensor.
Measuring electrodes 7 are positioned both in the lower chamber 9 and on the outside of the device. In this specific configuration, stainless-steel electrodes are used, but other materials are also possible.
By means of the measuring electrodes 7 arranged on the inside in the chamber 9 and on the outside on the housing 1, an ECG can be measured between fingers of the left and right hand. Likewise, various bioimpedance measurements are possible. By combining different measuring electrodes 7, the following bioimpedance measurements can be carried out in the configuration shown, for example:
   Bioimpedance measurement between the left and right index finger.
   Bioimpedance measurement between the right index finger and the right thumb.
A temperature sensor 8 is positioned on the outside, which measures the finger temperature when it comes into contact with a finger. For example, the temperature sensor 8 may also be integrated in the chamber 9.

The order and relative positioning of the sensors can correspond to the positioning in FIGS. 1 a-f and 2 a-d, but can also be adapted for specific applications. For example, the optical module 11 could also be positioned between the two electrodes 7 of the inner finger support.

The multifunctional measuring device is operated by a battery or rechargeable battery and comprises a plurality of measuring units. In variants of the measuring device without a docking station, external interfaces are integrated directly into the measuring device. The basic shape of an embodiment of the measuring device is rectangular (for example, length×width×height (approx.): 7 cm×4.5 cm×3.5 cm, weight: 85 g), but the exact shape differs from a rectangle for ergonomic and functional reasons. For example, the measuring device has to be able to open and the corners of the housing 1 are rounded to prevent any sharp edges.

FIG. 3 a-b show an exemplary measuring process. The user holds the measuring device in their hands and inserts their left index finger into the openable measuring device. The remaining fingers hold the measuring device, with measuring units also being positioned on the outside of the housing 1, which are provided for the right index finger and the right thumb in this case.

By means of the outer and inner measuring units of the measuring device, various types of measurement are possible on the fingers:
Optical measurements: Measurements of the transmission and the diffuse reflection at different wavelengths on the left index finger.
Electrical measurements: ECG measurements (between the left and right index finger) and various bioimpedance measurements (likewise between the left and right index finger, and between the right index finger and the right thumb).
Temperature measurement: Measurements of the finger temperature on the right index finger.

The measurement is also possible on other fingers. For example, the measurement could be taken on the middle finger instead of the index finger, or the left hand and right hands could be swapped over.

In order to read out and process the data generated by the measuring device, the invention has a microcontroller. Depending on the parameters to be measured, the microcontroller can execute different measuring programs in the process which differ in terms of the measuring units used, and the duration and order of the measuring processes that are carried out. Depending on the intended application and the user parameters to be determined, the duration of a measuring program of this kind is between a few seconds and several minutes.

FIGS. 3 and 4 show how the typical sequence of a measuring process that consists of executing the measuring program and subsequently calculating the results using the measuring device according to the invention may look. The typical sequence comprises the following steps:
The user takes the measuring unit in their hand and selects a measurement using a menu shown in the display 2b of the measuring device by means of two control elements 2c (buttons).
The user is prompted to insert their left index finger into the measuring device. The fingers of their right hand are placed onto the sensors arranged on the outside of the housing 1 and the measuring device is held as shown in FIGS. 3a and b.
Using an optical, electrical and/or temperature measurement, the measuring device identifies that the finger has been inserted and/or that the fingers are in contact with the external sensors.
A measuring program stored in the microcontroller software that has a fixed duration is started and individual measuring units of the measuring device are actuated and read out in a predetermined manner.
The measured data are analyzed and specific parameters are calculated for the individual measured signals.
On the basis of the measured parameters, further, optionally statistical analyses of the measured data can be carried out, which also take into account the results of earlier measurements.
The result of the measurement is displayed to the user and the results are saved. The displayed result may either be a parameter that is derived directly from the measurement or a parameter determined from a further, possibly statistical analysis.

The data processing and analysis can either be carried out by the microcontroller in the device, or the data are transmitted to an external data-processing unit and processed and evaluated therein. In this case, the data can be transmitted in a wired or also wireless manner, for example over Bluetooth or the like.

It is thus also possible to implement the user interface for operating the measuring device on the external data-processing unit, for example a smartphone or the like.

Irrespective of the device variant, the measuring device is operated by a battery or rechargeable battery in order to increase the electrical safety for the user.

The invention has various circuit parts for implementing the measuring function, analysis and storage, and optionally the transfer, of the data, as well as user interaction and monitoring of the device. In a possible configuration, the various circuit parts can be roughly divided into an analogue circuit part and a digital circuit part. The electronic concept of the measuring device is shown in FIG. 5 for this case.

Here, the analogue circuit part contains the electronics necessary for reading out the measuring units and the analogue processing of the measured signals (ECG, bioimpedance, temperature and optics circuits). Depending on the embodiment of the measuring device, these circuit parts may contain one or more analogue filter stages, but do not have to. The data from the measuring units are digitized for the further digital processing by one or more multi-channel ADCs (analogue-digital converters). The active parts of the measuring units (actuating the LEDs, generating the alternating current for the bioimpedance measurements) are likewise found in the analogue circuit part.

In the configuration shown, the digital circuit part comprises the microcontroller required for controlling the electronics and processing the measured data, together with additional memories that are both volatile and persistent. In addition, the controller for the control elements and the display are found in this circuit part. In addition, an optional Bluetooth chip and additional electronics for monitoring the device status, including the charging status of the battery or rechargeable battery, can be implemented in this circuit part.

In embodiments of the invention in which the measured data and/or results are transferred to other devices, however, not all of these circuit parts have to be provided: For example, it is conceivable for the persistent memory outside the microcontroller to be dispensed with if measured results are saved on another device.

By contrast, in device variants without a docking station, the circuit has to be supplemented with a charging circuit for the rechargeable battery and an electrical protective circuit, where necessary, in order to increase the electrical safety. In device variants with a docking station, the charging circuit for charging the rechargeable battery can be implemented completely in the docking station, meaning that the volume of the circuit in the measuring device can be reduced. In this case, communication with external devices via wired interfaces such as USB likewise takes place solely via the docking station.

### Concept of the Microcontroller Software:

The software saved on the microcontroller allows for the measuring process, the analysis of the measured data, as well as the interaction of the measuring device with the user and the environment via corresponding interfaces and protocols (e.g. USB and Bluetooth).

The possible main tasks of the firmware are:
Carrying out a device self-test when switching on the measuring device, such that the probability of incorrect measurements due to hardware defects can be reduced.
Interacting with the user via the control elements 2b and the display 2c (based on a graphical menu), also for displaying instructions for the measuring process.
Executing different types of measuring program which combine the different measuring units in different ways.
Actuating and reading out the measuring electronics.
Analyzing the raw data and optionally calculating additional parameters using statistical methods.
The result can be displayed both as a numerical value and graphically. One example of the latter would be the display of a colored bar, which uses different colors to show the normal range of a parameter and values outside this normal range. In this case, the measured result can be displayed and classified for the user by an arrow being shown which refers to a specific position within the bar.
Saving and loading user inputs, configuration files and measured data.
Displaying earlier measured values.
Communicating with the docking station and with the environment via the external interfaces, which are implemented via the docking station if applicable (e.g. USB interface).
Additional auxiliary functions such as language selection, display of device information, etc.
Not all the listed functions (e.g. analysis of the raw data) have to be implemented in the microcontroller software. Sub-steps can also be swapped to another device.
The general approach when executing measuring programs and analyzing the resulting data will be discussed in greater detail in the following.

### Execution of Predetermined Measuring Programs:

The measuring device according to the invention allows different measuring programs defined in the microcontroller software to be executed. These measuring programs can be differentiated by the duration and type of partial measurements that are carried out and/or the sensor system used. The measuring program used depends on the respective target parameters. Examples of possible target parameters and associated measuring programs are as follows:
Calculating the user's heart rate or other ECG parameters from an ECG measurement.
Determining an indicator of the user's pulse wave velocity by simultaneously measuring a photoplethysmogram and an ECG.
Determining the arterial oxygen saturation by optical measurements on the finger at different wavelengths.
Measuring the bioimpedance along the measuring paths predetermined by the measuring electrodes 7 at a certain frequency (e.g. 50 kHz). Measurements using multiple frequencies, including passing through a certain frequency range, are also conceivable.
Measuring the user's finger temperature using the temperature sensor 8 of the measuring device that is in contact with the finger.

The above-mentioned measuring programs set out by way of example can also be combined with one another, such that several target parameters can be determined within the same measuring program.

It should be noted that certain target parameters can be determined using a plurality of measuring units, such that the measured results of the individual measurements can be compared with one another and checked for plausibility. In particular, the determination can also be carried out simultaneously, depending on the measuring units used. As a result, the reliability of the results is increased. Examples of multiple determination processes of this kind are as follows:
Determining the heart rate from the ECG measurement and from a photoplethysmographic measurement using the optical measuring units.
Determining photoplethysmographic parameters from the optical measurements (photoplethysmography) and from the impedance measurements (impedance plethysmography).
Determining oximetric parameters from the optical measurements and from the thermal measurements.

For the end user, the microcontroller software can be configured such that either a predetermined measuring program is executed or a selection can be made between different measuring programs.

### Analysis of the Measured Data:

In principle, the analysis of the measured data can be divided into two main steps, in conceptual terms:
1. Analyzing the measured signals and deriving specific parameters from the measured signals.
2. Applying further, optionally statistical methods for calculating further parameters that are not accessible to the direct measurement.

Depending on the application, both steps of the analysis do not have to be implemented. If, for example, only the user's heart rate is measured and displayed, then it is sufficient to directly derive this from the measured signals. A further statistical analysis is not required.

For the analysis of the measured signals, various functions that are specifically adapted to the characteristics of the relevant measured signal and for the calculation of the target parameters are performed in the microcontroller software. Such functions include:
Pre-processing the measured signals (e.g. baseline correction, noise suppression).
Calculating statistical characteristics of the measured signals.
Calculating characteristic points in the measured signals.
Evaluating the signal shapes in signals having a characteristic progression over time (e.g. ECG).
Combining the information from various measured signals (e.g. determining the arterial oxygen saturation and the oxygen consumption in the arterial/venous tissues on the basis of the different absorption characteristics of the finger at different wavelengths).

Not all of these steps have to be implemented, depending on the application.

The parameters obtained by the measuring device are also standardized in different ways and are weighted according to both the physiological and physical calibration. The relationship between the parameters and e.g. the blood-glucose level can be established by means of mathematical models and confirmed using biostatistics. To do this, the parameters of the individual signals and possible combined parameters can be used for a selected statistical method.

The data can also be saved in an external database, via devices for data transfer. The result calculated by means of the statistical method can then be displayed to the user and can optionally be saved in the internal memory of the measuring device or a database.

### Variants of the Measuring Device:

The steps set out in the preceding sections, including determining the blood-glucose level, can take place directly on the measuring device (stand-alone variant). Alternatively, the analysis of the data can also be swapped to another device or a server (remote variant), for example if this is too computationally intensive for the measuring device. In this case, individual process steps or all the process steps that take place after the measured data is gathered, including saving the data, take place on another device, e.g. a server from the manufacturer or another contractually bound organization.

The measuring device is connected to another device, such as a PC or mobile telephone, via wireless communication, for example by means of Bluetooth. A specific application, which communicates with the measuring device, is executed on the other device. In this case, an essential task of this application consists in transferring the measured data to a server over an Internet connection. This may take place in the form of streaming during the measurement or by sending the complete set of measured data after the measurement is complete.

The measured data are then analyzed on the server. The result of the measurement calculated on the server can then be displayed on the external device or the measuring device.

Furthermore, the application running on the external device can expand the functionality of the measuring device by a graphical display of the history of the measured values or an export of the measured results for further use being implemented, for example.

### Expansion Options of the Invention:

The measuring device according to the invention can be expanded in a number of ways without altering the core concept of the invention. The general options for expansion and alteration already explained above in particular include:
The number, type and position of the sensors of the different measuring units.
The shape and size of the housing 1.
The measuring programs executed and the target parameters calculated from the measured data.
The implementation of stand-alone and remote variants of the measuring device which e.g. use wireless communication options such as Bluetooth.
The use of the measuring device with and without a docking station, and with a permanently installed or exchangeable rechargeable battery. In variants without a docking station, external interfaces such as USB can be directly integrated in the device.

Additional, specific expansion options are described in the following. The expansion options are grouped thematically here.

### Expansion of the ECG and Bioimpedance Measurements:

Additional electrodes can be added to the measuring device for further bioimpedance measurements or the existing electrodes can be used for other measurements, e.g.:
Measuring the impedance between the left index finger and the right thumb using the existing electrodes.
Adding two additional electrodes in the inner finger cavity or the outside of the measuring device such that the impedance can be measured on a single finger (local measurement).
Two additional electrodes on the outside such that the impedance between the two thumbs can be measured.
External connections for further (adhesive) electrodes comprising cables, such that the impedance can also be measured at body parts other than the fingers (e.g. on the arm).
Additional electrodes on the rear of the device, such that the measuring device can be pressed against the body and the bioimpedance between the inserted finger and the corresponding body part can be measured.

Additional electrodes can be added or the existing electrodes can be used differently in order to carry out an alternative ECG measurement:
With another electrode on the outside of the measuring device, the ECG measurement could also be carried out on the thumbs.
A plurality of electrodes can be interconnected for the ECG measurement in order to enlarge the effectively used electrode surface area (e.g. interconnecting the two inner electrodes on the left index finger and interconnecting the two external electrodes on the right index finger).
Connections for other external electrodes can also be added such that multi-channel ECG measurements can be carried out or a “right leg drive” can be used to reduce common-mode interference.
Other electrodes can be added such that the electrodes used for the ECG measurement and the bioimpedance measurement are completely disconnected in terms of circuitry.

The distance between the current-feeding and voltage-measuring electrodes 7 for the bioimpedance can be varied.

The geometry of the electrodes can be altered:
The electrodes can be reduced in size or enlarged.
The shape of the electrodes 7 can be altered (e.g. use of circular electrodes).
In order to effectively utilize the space in the chamber 9, one of the electrodes 7 in the chamber 9 can be shaped such that the optical module 11 is in a recess within the electrode.

The material of the electrodes can be altered (e.g. use of special types of steel or a completely different material).

The surface of the electrodes can be altered (e.g. use of smooth or roughened electrodes).

In order to improve the ECG or bioimpedance measurements, a liquid (also water) or a form of contact gel can be applied to the electrodes or to the fingers.

Instead of permanently installed electrodes, exchangeable electrodes can also be used. For example, in this case Ag/AgCl electrodes can be used, which are inserted into the device just before the measurement and are removed again after the measurement.

The bioimpedance measurement may be carried out in a bipolar, tripolar or tetrapolar manner. A matrix-shaped arrangement of electrodes is also possible, in which measurements can be carried out using different combinations of electrodes.

The bioimpedance measurements can be carried out both with a constant current and with a constant voltage.

In order to identify problems with the bioimpedance measurement (e.g. due to excessively high transition resistances on the finger), the current actually flowing in the bioimpedance measurement can be measured by expansions to the bioimpedance circuit. In addition, the progression over time of the current (e.g. sinusoidal shape) can be checked.

### Expansion of the Optical Measuring Units:

The number of optical sensors can be altered; for example, the optical sensor in the upper part of the chamber could be dispensed with or another optical sensor could be added at a certain distance from the existing sensor. With another sensor, propagation-time differences could be determined or tissue properties could be spatially resolved.
It would likewise be conceivable to use an array or a matrix-shaped arrangement of optical sensors in the upper part of the chamber instead of individual optical sensors, such that optical tissue properties can be spatially resolved, for example.
Another optical module could be used in the lower part of the inner finger support, which for example contains light sources having wavelengths that are optimized for a specific application.
Likewise, an array or a matrix-shaped arrangement of optical sensors could be integrated in the optical module of the lower finger support.
The properties of the optical sensors may be adapted depending on the intended application; for example, sensors having different active areas or different sensitivities in certain wavelength ranges could be used.
The distance between the optical module and the opposite additional optical sensor can be varied such that the light path through the tissue is longer or shorter, or the light has passed through other portions of tissues before it is detected.
The measuring device has a plurality of optical sensors which are positioned along the finger. Since the distance between the sensors is known, an indicator of the pulse wave velocity can be calculated without additional ECG measurement from the time difference with which the different optical sensors detect the change in the tissue absorption due to the pulsation of the arterial blood. In this case, it would be advantageous to select the distance between the optical sensors in the measuring system to be as great as possible, so that the time difference to be measured is also as great as possible.
The distance between the light sources and the optical sensors can be changed such that the penetration depth of the light into the tissue changes.
The intensity of the light sources can be varied and can be adapted to the characteristics of the user, e.g. depending on the finger thickness, by means of a gain factor.
In addition to the finger measurements, empty measurements (measurements of the intensity of the light sources without a finger inserted) can also be carried out in the measuring device. If, however, the optical module contains a sensor system for determining the intensity of the light sources, such measurements can be dispensed with.
In the case of an empty measurement for standardizing intensity, the empty measurement can be carried out at a different amplification to the finger measurement, such that the optical sensors are prevented from becoming saturated during the empty measurement.
The light sources can be operated using multiplexing or modulation methods instead of being activated sequentially. The resulting signal from the optical sensors then has to be accordingly split into the components of the different light sources using demultiplexing or demodulation.
The light sources in the optics module may also be, besides LEDs, other light sources, e.g. diode lasers or quantum-dot LEDs (QLEDs).
Multiplexing or modulation when operating a plurality of light sources could be dispensed with if the optical sensors were implemented in the form of a miniaturized spectrometer.

### Expansion of the Temperature Measurements:

The number of temperature sensors can be varied. For example, separate temperature sensors may be used for determining the housing temperature, the temperature of the electronics, and the temperature of the finger.
Depending on the type and position of the temperature sensors, different types of temperature measurement can be carried out, e.g. with and without direct contact with the sensor: When there is direct contact between the sensor and the finger, the temperature is determined by thermal conduction. It is, however, also conceivable for the thermal radiation of the finger to be measured or the body temperature to be measured by a contactless measurement, for example.
If the temperature of the finger is to be determined on the basis of the thermal radiation emitted thereby, a radiation-sensitive temperature sensor can be integrated in a depression in the housing such that thermal radiation from the finger can reach the temperature sensor, but there is no direct contact with the finger. In this case, in order to improve the accuracy of the temperature determination based on the radiation measurement, a second, structurally identical temperature sensor may be used, which is shielded both against direct contact with the finger and against thermal radiation from the finger. The measurement by the second temperature sensor can then act as a reference measurement for the first temperature sensor. The second temperature sensor can e.g. be shielded by integrating the temperature sensor in a closed cavity within the housing wall.

### Expansion of the Device Mechanics:

The spring mechanism can be altered such that the spring is exchangeable or can be set with regard to the spring strength, such that the same pressure can be produced for users with fingers of different thicknesses.
Instead of using a rear wall at the end of the finger cavity for positioning the finger, a palpable raised portion can also be integrated in the contact surface of the chamber, which indicates the correct finger position to the user.
The measuring device can be designed such that it is water-tight and dust-tight. In addition, the housing can be modified such that it withstands being dropped from a height.
The shape of the housing can be varied; for example, an oval housing is also conceivable. The exact length, width, height, and color, the housing material used or the surface structure of the housing material likewise do not alter the invention.
The geometry of the finger support, for example the radius of curvature or the length of the finger support, can be changed such that the device can equally be used by different user groups, e.g. children and adults.

### Improving the Handling of the Measuring Device:

In order to make it easier to handle the measuring device and simultaneously make possible a reproducible position of the fingers on the external sensors, stoppers can be attached to the outside of the measuring device. Alternatively, the external sensors can also be integrated in planar cavities, which predetermine the finger position.

### Expansion of the Measuring Programs:

A development of the invention provides that the measuring program used for capturing the measured data does not have a fixed duration, but is dynamically adapted to the quality of the measured data and/or the purpose of the analysis. For example, it is conceivable that an ECG measurement is taken until a certain number of ECG pulses have been measured, rather than predetermining a fixed measuring duration. It is likewise conceivable for the signal-to-noise ratio of the measured signal to be taken into account in different measurements and for a measurement to be taken for longer in users with a poor signal-to-noise ratio than in users with a good signal-to-noise ratio.
For certain applications, it may be advantageous for the measuring device to differentiate between training measurements (no result displayed) and test measurements (with result displayed). The training measurements may be used to familiarize the user with handling the device, for example.

### Expansion of the Data Analysis:

A development of the invention provides for using machine-learning methods in one or both of the above-mentioned conceptual analysis steps (consisting of the parameter extraction from the measured data and the use of statistical methods for the model-based calculation of further parameters).
In this case, different machine-learning methods can be used and even combined for different sub-steps. The machine-learning methods that can be used here in particular include neural networks, support vector machines and decision trees, including random forests, or methods derived therefrom.
The machine-learning methods may for example assist the calculation of the parameters from the measured signals or, for example, may also partially or completely replace the use of conventional signal-processing methods. In the same way, the machine-learning methods can be used to generate, on the basis of training data, models for calculating other parameters that are not directly accessible to the measurement.
Likewise, machine-learning methods can also be used to execute a plurality of analysis steps simultaneously. This in particular involves the fact that the steps for parameter extraction from the raw data and the model-based calculation of further parameters can be combined. For this purpose, the use of deep neural networks (what is known as deep learning) is provided, since deep neural networks can automate the process of parameter extraction such that said parameters no longer need to be explicitly defined and calculated.
Depending on the complexity of the models used, they can either be integrated directly in the software of the measuring device and evaluated by the software of the microcontroller, or can be evaluated on another device to which the measured data or parameters are transmitted by the means for data transfer.
   Expanded Utilization of the Data from the Measuring Device:
Instead of displaying individual parameters on the measuring device as a result, measured data can also be transmitted to another device, e.g. a personal computer, such that the measured signals (e.g. ECG) can be viewed and evaluated directly, by medical personnel, for example, by means of a corresponding application. In the same way, the history of various physiological parameters can be transmitted in order to make it possible for medical personnel to evaluate the development of a user's state of health over a longer period of time, for example.
The user can be offered various measuring programs, by means of which various parameters (e.g. heart rate, oxygen consumption, blood pressure or blood-glucose level) can be measured depending on need and interest. Separate histories can be compiled for the measured parameters and can be displayed to the user.
If the results (e.g. heart rate or blood glucose) generated by the measuring device are transmitted to another device, e.g. a personal computer or a smartphone, this device can additionally be connected to a database in which the user saves information relating to their lifestyle habits (type and frequency of meal times, exercise, etc.). By linking measured results and information relating to lifestyle habits, the effects of these lifestyle habits on the user, e.g. the effect of the type of food on their measured blood glucose, can be monitored. In reverse, the additionally saved information can also be used to improve the model-based calculation of parameters.
For relationships between measured data and target parameters that are only applicable to certain groups of people, a user can also be assigned to such a group of people on the basis of the measured parameters by applying a statistical method (e.g. clustering).
Based on a history of measured parameters (e.g. after a training phase), the user can be recognized or identified on the basis of their measured values by means of statistical methods. As a result, the measuring device can be used in a personalized manner. In particular when the measuring device is configured in a user-specific manner, a user can also be prevented from accidentally using an incorrect configuration.
For certain applications, it is likewise conceivable for a relationship between the measured parameters and a target parameter, for example blood glucose, to be developed for certain users in cooperation with medically trained personnel and for this relationship to then be saved in the measuring device for these specific users in the form of a configuration file.

### Improving the Ease of Use:

The display of the measuring device can be anti-glare, such that the display of the measuring device can be easily read even in very bright conditions.
Status LEDs can be integrated in the housing of the measuring device, which display the charging status of the battery or rechargeable battery, for example.
The orientation of the display of the measuring device can be changed (for example, installing the display longitudinally instead of transversely). In addition, changes in the orientation of the device could be identified by means of a gyroscopic sensor, such that the orientation of the display is automatically changed by the software.
The measuring device can be expanded such that acoustic signals, for example for the end of the measurement, can be generated.
It is likewise conceivable for the measuring device to be expanded with speech output, such that the result or instruction can be communicated to the user by speech output. As a result, the usability of the device would be improved for visually impaired people.
For information purposes, measured data can be displayed to the user on the integrated display of the measuring device during the measurement.
   Expansion of the Use of Wireless Communication, e.g. Bluetooth:
When the measuring device is connected to another device, e.g. a smartphone, via wireless communication, the display of the smartphone can be used as a supplementary display for the measuring device or as a complete replacement for the display of the measuring device. Since the display of a smartphone is typically considerably larger than the display of the measuring device, in this case more detailed measured statistics can be displayed or the result can be displayed in a larger font, for example, with the latter being helpful in particular for visually impaired people. The smartphone or another external device can also be used completely as a user interface for the measuring device, such that the control elements that are integrated in the measuring device can be reduced.
The software of the measuring device and the communication protocols thereof can be adapted such that they are compatible with standardized communication protocols, e.g. in the network of a hospital, and the results from the measuring device can be transferred directly into this network.
Software updates or updates of configuration data of the measuring device, for example, can also be carried out via wireless communication by being transmitted to the measuring device from a smartphone, for example.
If the docking station has its own memory, the wireless-communication capability of the measuring device can also be used as a data-transmission path between the docking station and the environment. In this case, via the wireless connection, configuration or calibration data can be transmitted to the docking station and stored in its memory.
It is conceivable that the measuring device first has to be authenticated on a server operated by the manufacturer over wireless communication (using the Internet connection of a smartphone where applicable, for example) before measurements are carried out, e.g. for safety reasons. The authentication could e.g. be based on exchanging special cryptographically signed keys.
The described concept involving authentication of the measuring device on a server could also be used to implement a payment system for the measuring system.

### Various Expansion Options:

For certain applications of the measuring device, e.g. in a hospital, it is conceivable to reduce the electronics in the measuring device such that only the part of the electronics required for reading out the sensor data and for subsequently digitizing the data is found in the measuring device. The measured data could then be transmitted in a wired or wireless manner to a control and evaluation unit, which analyses the data and displays the data or the results calculated from these data on a monitor.
The device and multi-sensor concept can be transmitted to a watch or smartwatch, or a ring, which the user can wear all the time. As a result, blood-glucose measurements can be simply integrated into the user's daily routine without them having to carry around an additional device.
The rechargeable battery of the measuring device may be permanently installed or exchangeable. In the latter case, the rechargeable battery could also be charged by an external charging device rather than by the docking station.
The docking station may have one or more of the following functions: Charging the rechargeable battery of the measuring device by an external connection of the docking station (e.g. USB).
Providing additional wired interfaces, e.g. USB. In addition to transferring measured data, these interfaces can also e.g. be used to carry out software updates or to update configuration data.
Electrically protecting the user by electrically insulating (galvanically isolating) the current lines and data lines from the mains power.
Additionally protecting the user by implementing a mechanism which prevents the electrodes of the measuring device from being able to be touched while the measuring device is in the docking station and is being charged.
Providing a transport case in which the measuring device can be safely transported.
Testing or calibrating the measuring device if means for testing or calibrating one or more sensor units of the measuring device are integrated in the docking station.

One advantage of the device variant having a docking station is for example that the charging circuit and the electrical protective circuit do not have to be integrated in the measuring device, and therefore the volume of the electrical protective circuit in the measuring device can be reduced in size.

### LIST OF REFERENCE SIGNS

1 housing
2 upper shell
2a ridge on the upper shell
2b display
2c control elements
3 lower shell
3a depression in the lower shell
4 hinge mechanism
5 connector
6 metal contacts
7 electrodes
8 temperature sensor
9 chamber
9a soft material
10 wall
11 optical module
12 light source
13 additional optical sensor

1. A multifunctional measuring device, comprising a housing (1) having an upper shell (2) and a lower shell (3), which are movable relative to one another by means of a hinge mechanism (4) and comprise cavities which correspond to one another, wherein the cavities form a chamber (9) accessible from the outside for receiving a human finger, wherein an optical measuring unit having an optical module (11), which comprises at least one light source (12) and at least one sensor, is arranged in the chamber (9), and wherein means for data evaluation and/or data transfer are integrated in or on the housing, wherein at least one electrical measuring unit is provided, comprising at least two measuring electrodes (7) in the chamber (9) and/or on the outside of the housing (1).

2. Multifunctional measuring device according to claim 1, wherein at least one temperature-measuring unit is arranged in and/or on the housing (1).

3. Multifunctional measuring device according to claim 1, wherein at least one additional optical sensor (13) and/or one additional light source is arranged in the chamber (9) opposite the optical module (10).

4. Multifunctional measuring device according to claim 1, wherein the hinge mechanism is provided with a return mechanism.

5. Multifunctional measuring device according to claim 1, wherein a microcontroller is arranged in the housing (1) for data evaluation.

6. Multifunctional measuring device according to claim 1, wherein the means for data transfer have a wireless interface.

7. Multifunctional measuring device according to claim 1, wherein devices for positioning the individual fingers are provided such that the fingers are always in the same position during the measuring process.

8. Multifunctional measuring device according to claim 1, wherein an accelerometer is integrated.

9. Multifunctional measuring device according to claim 1, wherein a gyroscope is integrated.

10. Multifunctional measuring device according to claim 1, wherein additional sensors are integrated for measuring the air pressure, humidity and/or the ambient temperature.

11. Multifunctional measuring device according to claim 1, wherein pressure sensors are integrated for measuring the contact pressure of the finger.

12. Multifunctional measuring device according to claim 1, wherein connections for additional external sensor systems are arranged on the housing (1).

13. A method for carrying out a measurement using a multifunctional measuring device according to claim 1, wherein one or more physiological parameters are determined by executing predetermined measuring programs by using and/or combining a plurality of measuring units.

14. Method for carrying out a measurement using a multifunctional measuring device according to claim 13, wherein additional parameters that are otherwise not accessible to a non-invasive measurement are determined from the measured signals using statistical methods and/or machine-learning methods.
